(19) United States
(12) Patent Application Publication
(10) Pub. No.: US 2022/0246238 A1
(43) Pub. Date: Aug. 4, 2022

(54) ATOMIC SEQUENCE REARRANGEMENT METHOD

(71) Applicant: SHENZHEN JINGTAI TECHNOLOGY CO., LTD., Guangdong (CN)

(72) Inventors: Chao CHANG, Guangdong (CN); Jian MA, Guangdong (CN); Lipeng LAI, Guangdong (CN); Shuhao WEN, Guangdong (CN)

(73) Assignee: SHENZHEN JINGTAI TECHNOLOGY CO., LTD., Guangdong (CN)

(21) Appl. No.: 17/617,594

(22) PCT Filed: May 29, 2020
(86) PCT No.: PCT/CN2020/093287

§ 371 Date: Dec. 9, 2021

(51) Int. Cl. G16B 30/10; G16B 15/00; G16B 5/00

(52) U.S. Cl. CPC G16B 30/10; G16B 5/00; G16B 15/00

(57) ABSTRACT

A method of atomic sequence rearrangement, includes the following steps. Topological rearrangement: the atomic sequence of the target structure is rearranged referring to the reference structure using the two-dimensional topological rearrangement method. Judgment of equivalent atoms: judge the equivalent atoms in the topological structure. Measuring and marking: mark the atomic chiral information of the rearranged structure and the reference structure. Second rearrangement: refer to the reference structure for the second rearrangement of the atomic sequence for the rearranged structure.

### TECHNICAL FIELD

The invention pertains to the pre-processing of molecular force field energy calculation, in particular to a method of atomic sequence rearrangement.

### BACKGROUND TECHNOLOGY

Before calculating the force field energy of the structure, it is necessary to rearrange the atomic sequence. The current method of rearranging the atomic sequence is to rearrange the atoms through the topological comparison of the graph theory tool networkx, and the topological comparison only contains the 2D information of the structure, while the 3D features of the structure may cause the rearranged atomic sequence to be wrong. So after the topological comparison of the rearrangement of the atomic sequence, manual inspection is still required, this is inefficient.

For example, rearrange the atomic sequence of a structure which contains a symmetric aliphatic ring, as shown in FIG. 11. If only the 2D information of the structure is considered, the atomic sequences of the two structures are matched; but if the 3D information is considered, the atomic sequences of the two structures do not match as shown in FIG. 12. Therefore, if the atomic sequence is rearranged using the topological comparison method that only contains 2D information; the atomic sequence may not match.

### DESCRIPTION OF THE INVENTION

Based on this, it is necessary to provide an atomic sequence rearrangement method that can improve the accuracy of atomic sequence rearrangement.

A method of atomic sequence rearrangement, including:

Topological rearrangement: the atomic sequence of the target structure is rearranged referring to the reference structure using the two-dimensional topological rearrangement method.

Judgment of equivalent atoms: judge the equivalent atoms in the topological structure.

Measuring and marking: mark the atomic chiral information of the rearranged structure and the reference structure.

Second rearrangement: referring to the reference structure for the second rearrangement of the atomic sequence for the rearranged structure.

In a preferred embodiment, wherein the measuring and marking step as: marking the atomic sequence chiral information of the rearranged structure and the reference structure according to the measurement and marking method of atomic chirality.

In a preferred embodiment, the method for measuring and marking the atomic sequence chirality is: taking the central atom as a starting point, and take the dihedral angles of the atoms connected to the central atom in a clockwise direction, and the atoms taken must contain equivalent atoms, If the dihedral angle is greater than 0, the two topologically consistent atoms are marked as True and False in the order of taking the atoms. If the dihedral angle is less than 0, the two topologically consistent atoms are marked as False and True in the order of taking the atoms.

In a preferred embodiment, the atomic chirality is that, if the atomic sequence of the molecular structure does not overlap with the atomic sequence of its mirror structure, it is judged to have atomic sequence chirality.

In a preferred embodiment, if the atomic chirality is that, if the topological connection degree of the atom is greater than or equal to 3, it is judged to have atomic sequence chirality.

In a preferred embodiment, the measuring and marking step as: measuring the atomic sequence chirality of the central atom connecting two topologically equivalent atoms, and marking the measurement result on the equivalent non-hydrogen atom.

In a preferred embodiment, the judgment of equivalent atoms includes: judging topologically equivalent atoms through a list of adjacent atoms, and the list of adjacent atoms is generated according to the topological connections of atoms.

In a preferred embodiment, the equivalent atom is an atom having an equivalent adjacent atom list.

In a preferred embodiment, if there are two or more equivalent atoms among the atoms connected to the central atom, two atoms are arbitrarily selected as equivalent atoms.

In a preferred embodiment, the second rearrangement step includes: performing rearrangement of the original structure with atomicity information and the reference structure the second time to obtain a structure with the same atomic sequence of the reference structure.

The above-mentioned atomic rearrangement method will mark the atomic chiral information of the rearranged structure and the reference structure, and the rearranged structure will be referenced to the reference structure to perform a secondary rearrangement of the atomic sequence. Introducing atomic sequence chirality, and including part of the 3D information of the structure In the 2D topological atomic rearrangement, the atomic sequence rearrangement can fully consider the 3D information of the structure, avoid the disorder of the atomic sequence of the structure, and solve the problem of inconsistent atomic sequence, which will be beneficial to the subsequent accurate calculation of the force field energy of the structure.

### DESCRIPTION OF THE DRAWINGS

FIG. 1 is a flowchart of the atomic rearrangement method according to an embodiment of the present invention.

FIG. 2 is a schematic diagram of the adjacent atom list according to an embodiment of the present invention.

FIG. 3 is a schematic diagram of equivalent atoms in an embodiment of the present invention.

FIG. 4 is a schematic diagram of the atomic sequence chirality according to an embodiment of the present invention.

FIG. 5 is a schematic diagram of a secondary rearrangement according to an embodiment of the present invention.

FIG. 6 is a schematic diagram of the original structure requiring atomic sequence comparison according to another embodiment of the present invention.

FIG. 7 is a schematic diagram of a reference structure of another embodiment of the present invention.

FIG. 8 is a schematic diagram of a symmetrical six-membered ring of the original structure according to another embodiment of the present invention.

FIG. 9 is a schematic diagram of type A atomic sequence after referencing topological alignment according to another embodiment of the present invention.

FIG. 10 is a schematic diagram of type B atomic sequence after referencing topological alignment according to another embodiment of the present invention.

FIG. 11 is a schematic diagram of atomic sequence rearrangement of a structure containing an aliphatic ring considering only 2D information in the background art.

FIG. 12 is a schematic diagram of atomic sequence rearrangement considering 3D information in the background art.

### DESCRIPTION OF THE PREFERRED EMBODIMENTS

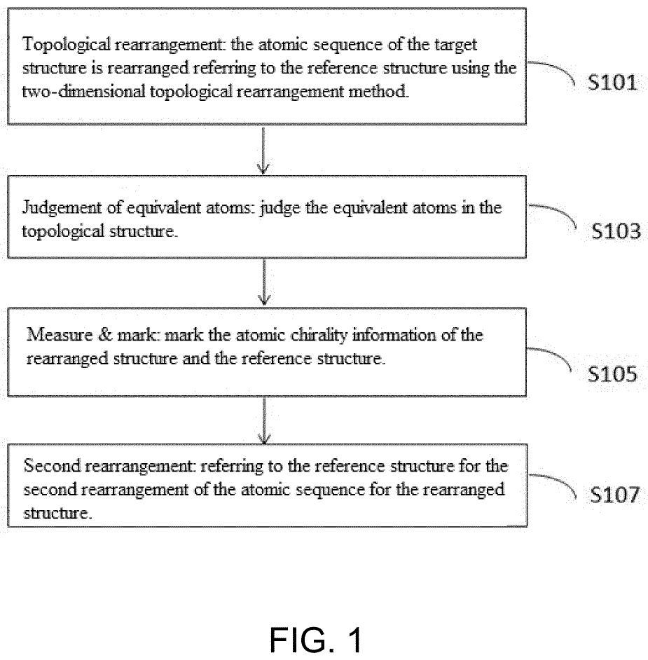

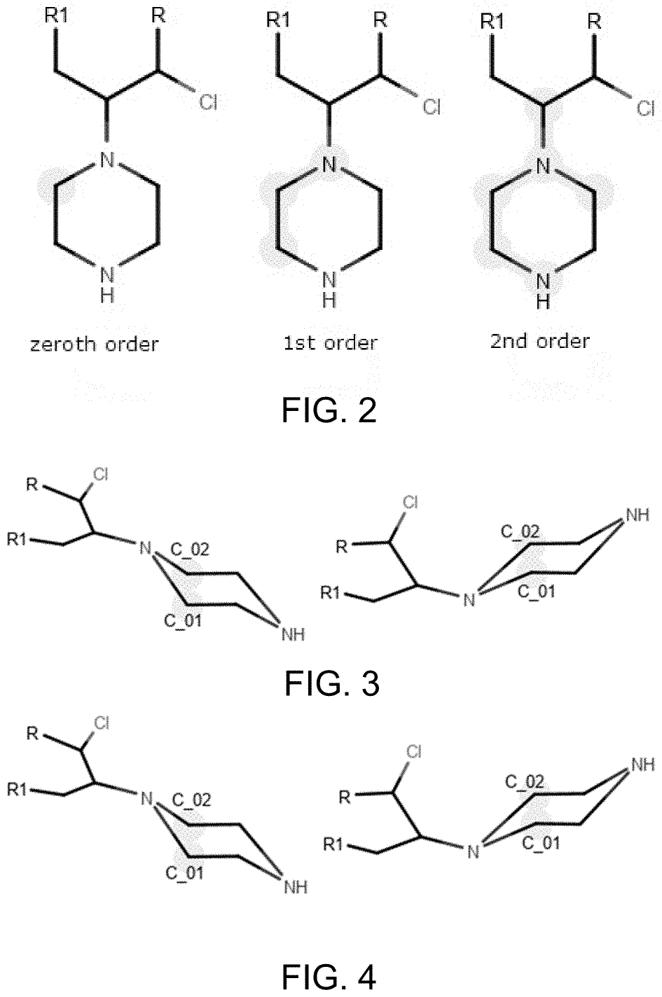

As shown in FIG. 1, the atomic sequence rearrangement method according to an embodiment of the present invention includes:

Step S101, Topological rearrangement: rearrange the atomic sequence of the target structure referring to the reference structure using a two-dimensional topological rearrangement method;

Step S103, equivalent atom judgment: judging the equivalent atoms in the topological structure;

Step S105, measuring and marking: marking the atomic sequence chiral information of the rearrangement structure and the reference structure;

Step S107, second rearrangement: the rearranged structure is subjected to a second rearrangement of atomic sequence referring to the reference structure.

Further, the topological rearrangement step of this embodiment: refer to the reference structure for the structure that the atomic sequence needed to be rearranged and use the is_isomorphic method of the isomorphism module in the networkx algorithm library to calculate the atomic correspondence relationship according to the two-dimensional topologies of the two structures, and rearrange the atomic sequence of the target structure according to the correspondence relationship. For the is_isomorphic method, please refer to: LP Cordella, P. Foggia, C. Sansone, M. Vento, “An Improved Algorithm for Matching Large Graphs”, 3rd IAPR-TC15 Workshop on Graph-based Representations in Pattern Recognition, Cuen, pp. 149-159, 2001.

Further, the measuring and marking step of this embodiment: marking the atomic sequence chiral information of the rearranged structure and the reference structure according to the measuring and marking method of atomic sequence chirality.

Further, the method for measuring and marking the chirality of the atomic sequence of this embodiment: taking the central atom as a starting point, and take the dihedral angles of the atoms connected to the central atom in a clockwise direction, and the atoms taken must contain equivalent atoms; If the dihedral angle is greater than 0, the two topologically consistent atoms are marked as True and False in the order of taking the atoms. If the dihedral angle is less than 0, the two topologically consistent atoms are marked as False and True in the order of taking the atoms.

Furthermore, the atomic chirality of this embodiment is that, if the atomic sequence of the molecular structure does not overlap with the atomic sequence of the mirror structure itself, it is judged to have atomic sequence chirality.

Furthermore, if the atomic chirality of this embodiment is that, if the topological connection degree of the atom is greater than or equal to 3, it is judged to have atomic sequence chirality.

Further, the measuring and marking step of this embodiment: measure the atomic sequence chirality of the central atom that connects two topologically equivalent atoms, and mark the measurement result on the equivalent non-hydrogen atom.

Further, the equivalent atom judgment in this embodiment includes: judging the topologically equivalent atoms through the adjacent atom list, which is generated according to the topological connection of the atoms.

Furthermore, the equivalent atom in this embodiment is an atom having an equivalent adjacent atom list.

Furthermore, if there are two or more equivalent atoms among the atoms connected to the central atom, two atoms are arbitrarily selected as equivalent atoms.

Further, the second rearrangement step of this embodiment includes: performing a second rearrangement of the original structure with atomic chiral information and the reference structure to obtain a structure consistent with the atomic sequence of the reference structure.

The present invention introduces the concept of atomic sequence chirality, and incorporates part of the 3D information of the structure into the 2D topological atomic sequence rearrangement. The atomic sequence rearrangement can fully consider the 3D information of the structure. Atomic sequence chirality: The atomic sequence of the molecular structure does not overlap with the atomic sequence of its mirror structure, indicating that the atom has atomic sequence chirality. Described from the perspective of topology, when the topological connectivity of an atom is greater than or equal to 3, it indicates that the atom has atomic sequence chirality. Adjacent atom list: Generate adjacent atom list according to the topological connection of atoms.

As shown in FIG. 3, equivalent atoms in an embodiment of the present invention. Atoms with a list of equivalent adjacent atoms are equivalent atoms. If there are more than 2 equivalent atoms among the atoms connected to the central atom, two atoms are arbitrarily selected as equivalent atoms.

As shown in FIG. 4, the method for measuring and marking the atomic sequence chirality in an embodiment of this invention: taking the central atom as a starting point, and take the dihedral angles of the atoms connected to the central atom in a clockwise direction, and the atoms taken must contain equivalent atoms; If the dihedral angle is greater than 0, the two topologically consistent atoms are marked as True and False in the order of taking the atoms. If the dihedral angle is less than 0, the two topologically consistent atoms are marked as False and True in the order of taking the atoms.

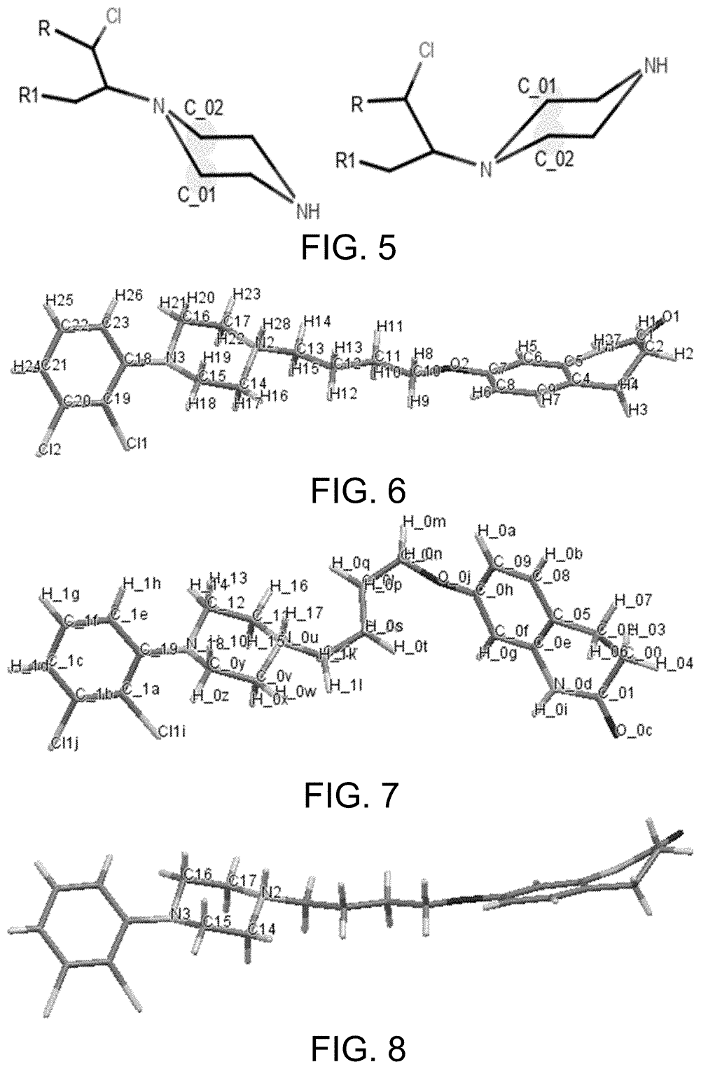

As shown in FIG. 5, in an embodiment of the present invention, the rearrangement structure with atomic sequence chiral information is referred to the reference structure for the second rearrangement of the atomic sequence.

By introducing the concept of atomic order chirality, part of the 3D information of the structure is included in the 2D topological atomic sequence rearrangement, so that the atomic sequence rearrangement can fully consider the 3D information of the structure and avoid disorder of the atomic sequence of the structure.

As shown in FIG. 6 to FIG. 10, another embodiment of the present invention describes that the original structure includes a symmetrical six-membered ring structure (as shown in FIG. 8).

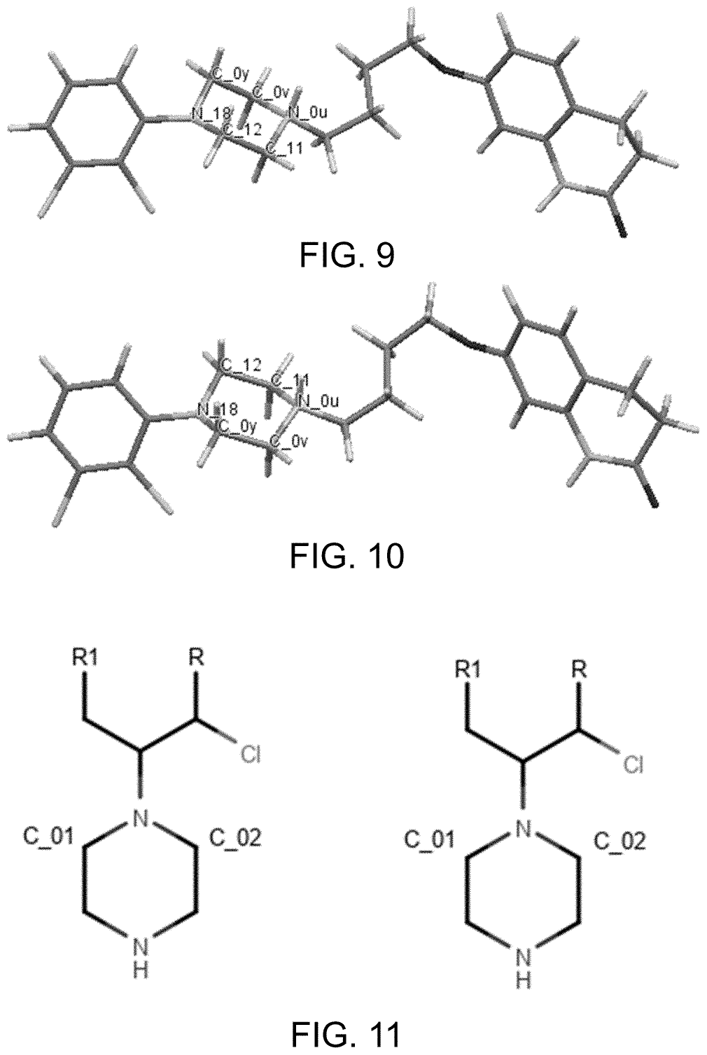

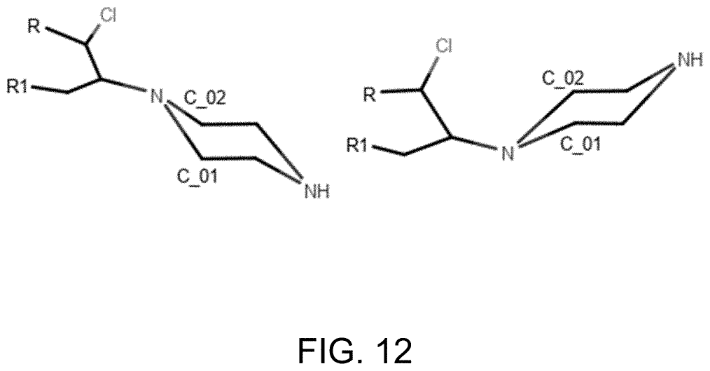

Due to the original structure containing a symmetric six-membered ring (as shown in FIG. 8), two types of atomic order markings, FIG. 9 and FIG. 10, may appear by the two-dimensional topological rearrangement method, of which only the type of FIG. 10 is consistent with the reference structure. After the improvement of the atomic sequence rearrangement method of the present invention, the rearrangement result will only contain type B (FIG. 10). The specific implementation method is as follows:

Rearrange the original structure (as shown in FIG. 6) with reference to the reference structure (as shown in FIG. 7) using the two-dimensional topology rearrangement method;

Judge topologically equivalent non-hydrogen atoms through the adjacent atom list. For example, in Table 1, the adjacent atoms of atom C_11 and C_0v are the same, and the adjacent atoms of atom C_12 and C_0y are the same.

The atomic sequence chirality is measured for the central atom connecting two topologically equivalent atoms, and the measurement result is marked on the equivalent non-hydrogen atom. For example, the atomic sequence chirality of the central atom N_18 in FIG. 9 and FIG. 10 is opposite. Therefore, the chiral marking of the equivalent atom is opposite;

The original structure with atomic sequence chiral information and the reference structure are rearranged for the second time and a structure consistent with the atomic sequence of the reference structure can be obtained.

The following Table 1 is the adjacent atom list of this embodiment

| Atom | Adjacent Atom |
|---|---|
| C_00 | 0C1CCCHH2CCCCHHHHNO |
| C_01 | 0C1CCNO2CCCCHHHNO |
| C_02 | 0C1CCCHH2CCCCCCHHHH |
| H_03 | 0H1CH2CCCHH |
| H_04 | 0H1CH2CCCHH |
| C_05 | 0C1CCCC2CCCCCCCHHHN |
| H_06 | 0H1CH2CCCHH |
| H_07 | 0H1CH2CCCHH |
| C_08 | 0C1CCCH2CCCCCCHH |
| C_09 | 0C1CCCH2CCCCCHHO |
| H_0a | 0H1CH2CCCH |
| H_0b | 0H1CH2CCCH |
| O_0c | 0O1CO2CCNO |
| N_0d | 0N1CCHN2CCCCCHNO |
| C_0e | 0C1CCCN2CCCCCCCHHN |
| C_0f | 0C1CCCH2CCCCCHNO |
| H_0g | 0H1CH2CCCH |
| C_0h | 0C1CCCO2CCCCCCHHO |
| H_0i | 0H1HN2CCHN |
| O_0j | 0O1CCO2CCCCCHHO |
| C_0k | 0C1CCHHO2CCCCHHHHO |
| C_0l | 0C1CCCHH2CCCCHHHHHHO |
| H_0m | 0H1CH2CCHHO |
| H_0n | 0H1CH2CCHHO |
| C_0o | 0C1CCCHH2CCCCHHHHHHN |
| H_0p | 0H1CH2CCCHH |
| H_0q | 0H1CH2CCCHH |
| C_0r | 0C1CCHHN2CCCCCHHHHHN |
| H_0s | 0H1CH2CCCHH |
| H_0t | 0H1CH2CCCHH |
| N_0u | 0N1CCCHN2CCCCCCHHHHHHHN |
| C_0v | 0C1CCHHN2CCCCHHHHHNN |
| H_0w | 0H1CH2CCHHN |
| H_0x | 0H1CH2CCHHN |
| C_0y | 0C1CCHHN2CCCCHHHHNN |
| H_0z | 0H1CH2CCHHN |
| H_10 | 0H1CH2CCHHN |
| C_11 | 0C1CCHHN2CCCCHHHHHNN |
| C_12 | 0C1CCHHN2CCCCHHHHNN |
| H_13 | 0H1CH2CCHHN |
| H_14 | 0H1CH2CCHHN |
| H_15 | 0H1CH2CCHHN |
| H_16 | 0H1CH2CCHHN |
| H_17 | 0H1HN2CCCHN |
| N_18 | 0N1CCCN2CCCCCCCHHHHN |
| C_19 | 0C1CCCN2CCCCCCCClHN |
| C_1a | 0C1CCCCl2CCCCCClClN |
| C_1b | 0C1CCCCl2CCCCCClClH |
| C_1c | 0C1CCCH2CCCCCClHH |
| H_1d | 0H1CH2CCCH |
| C_1e | 0C1CCCH2CCCCCHHN |
| C_1f | 0C1CCCH2CCCCCHHH |
| H_1g | 0H1CH2CCCH |
| H_1h | 0H1CH2CCCH |
| Cl1i | 0Cl1CCl2CCCCl |
| Cl1j | 0Cl1CCl2CCCCl |
| H_1k | 0H1CH2CCHHN |
| H_1l | 0H1CH2CCHHN |

The atomic sequence rearrangement method of the present invention is suitable for the pre-processing of the molecular structure force field energy calculation. By including part of the 3D information of the structure into the 2D topological atomic sequence rearrangement, the problem of atomic sequence inconsistency can be solved, thereby accurately calculating the force field energy of the structure.

Taking the above-mentioned ideal embodiment based on this application as enlightenment, and through the above description, relevant staff can make various changes and modifications without departing from the scope of the technical idea of this application. The technical scope of this application is not limited to the content in the specification, and its technical scope must be determined according to the scope of the claims.

Those skilled in the art should understand that the embodiments of the present application can be provided as a method, a system, or a computer program product. Therefore, this application may adopt the form of a complete hardware embodiment, a complete software embodiment, or an embodiment combining software and hardware. Moreover, this application may adopt the form of a computer program product implemented on one or more computer-usable storage media (including but not limited to disk storage, CD-ROM, optical storage, etc.) containing computer-usable program codes.

This application is described with reference to the method of embodiments of this invention and flowcharts and/or block diagrams of devices (systems), and computer program products. It should be understood that each process and/or block in the flowchart and/or block diagram, and the combination of processes and/or blocks in the flowchart and/or block diagram can be realized by computer program instructions. These computer program instructions can be provided to the processor of a general-purpose computer, a special-purpose computer, an embedded processor, or other programmable data processing equipment to generate a machine, so that the instructions executed by the processor of the computer or other programmable data processing equipment are generated. It is a device that realizes the functions specified in one process or multiple processes in the flowchart and/or one block or multiple blocks in the block diagram.

These computer program instructions can also be stored in a computer-readable memory that can direct a computer or other programmable data processing equipment to work in a specific manner, so that the instructions stored in the computer-readable memory produce an article of manufacture including the instruction device. The device implements the functions specified in one process or multiple processes in the flowchart and/or one block or multiple blocks in the block diagram.

These computer program instructions can also be loaded on a computer or other programmable data processing equipment, so that a series of operation steps are executed on the computer or other programmable equipment to produce computer-implemented processing, so as to execute on the computer or other programmable equipment. The instructions provide steps for implementing functions specified in a flow or multiple flows in the flowchart and/or a block or multiple blocks in the block diagram.

1. A method of atomic rearrangement, comprising:
topological rearrangement: an atomic sequence of a target structure being rearranged referring to a reference structure using a two-dimensional topological rearrangement method;
judgment of equivalent atoms: judging equivalent atoms in a topological structure;
measuring and marking: marking an atomic chiral information of a rearranged structure and the reference structure; and
second rearrangement: referring to the reference structure for the second rearrangement of the atomic sequence for the rearranged structure.

2. The atomic sequence rearrangement method according to claim 1, wherein the measuring and marking step is: marking the atomic sequence chiral information of the rearranged structure and the reference structure according to an atomic chirality measurement and marking method.

3. The atomic sequence rearrangement method according to claim 2, wherein the atomic sequence chirality measurement and marking method comprises: taking a central atom as a starting point, and taking dihedral angles of atoms connected to the central atom in a clockwise direction, wherein the selected atom must contain equivalent atoms; if the dihedral angle is greater than 0, marking the two atoms with a same topology as True and False in the order of taking the atoms; and if the dihedral angle is less than 0, marking the two atoms with the same topology as False and True in the order of taking the atoms.

4. The atomic sequence rearrangement method according to claim 2, wherein an atomic chirality is that, if the atomic sequence of a molecular structure does not overlap with the atomic sequence of its mirror structure, it is judged to have the atomic sequence chirality.

5. The atomic sequence rearrangement method according to claim 2, wherein an atomic chirality is that, if a topological connection degree of the atom is greater than or equal to 3, it is judged to have atomic sequence chirality.

6. The atomic sequence rearrangement method according to claim 1, wherein the measuring and marking step comprises: measuring the atomic sequence chirality of the central atom connecting two topologically equivalent atoms, and marking a measurement result on an equivalent non-hydrogen atom.

7. The atomic sequence rearrangement method according to claim 1, wherein the judgment of equivalent atoms step comprises: judging topologically equivalent atoms through a list of adjacent atoms, and the list of adjacent atoms being generated based on a topological connection of the atoms.

8. The atomic sequence rearrangement method according to claim 6, wherein the equivalent atom is an atom having a list of equivalent adjacent atoms.

9. The atomic sequence rearrangement method according to claim 6, wherein if there are two or more equivalent atoms among the atoms connected to the central atom, two atoms are arbitrarily selected as equivalent atoms.

10. The atomic sequence rearrangement method according to claim 2, wherein the second rearrangement step comprises: performing rearrangement of the original structure with atomicity information and the reference structure for a second time, obtaining a structure consistent with the atomic sequence of the reference structure.

11. The atomic sequence rearrangement method according to claim 2, wherein that the measuring and marking step comprises: measuring the atomic sequence chirality of the central atom connecting two topologically equivalent atoms, and marking a measurement result on an equivalent non-hydrogen atom.

12. The atomic sequence rearrangement method according to claim 3, wherein that the measuring and marking step comprises: measuring the atomic sequence chirality of the central atom connecting two topologically equivalent atoms, and marking a measurement result on an equivalent non-hydrogen atom.

13. The atomic sequence rearrangement method according to claim 4, wherein that the measuring and marking step comprises: measuring the atomic sequence chirality of the central atom connecting two topologically equivalent atoms, and marking a measurement result on an equivalent non-hydrogen atom.

14. The atomic sequence rearrangement method according to claim 5, wherein that the measuring and marking step comprises: measuring the atomic sequence chirality of the central atom connecting two topologically equivalent atoms, and marking a measurement result on an equivalent non-hydrogen atom.

15. The atomic sequence rearrangement method according to claim 2, wherein the judgment of equivalent atoms step comprises: judging topologically equivalent atoms through a list of adjacent atoms, and the list of adjacent atoms being generated based on a topological connection of the atoms.

16. The atomic sequence rearrangement method according to claim 3, wherein the judgment of equivalent atoms step comprises: judging topologically equivalent atoms through a list of adjacent atoms, and the list of adjacent atoms being generated based on a topological connection of the atoms.

17. The atomic sequence rearrangement method according to claim 4, wherein the judgment of equivalent atoms step comprises: judging topologically equivalent atoms through a list of adjacent atoms, and the list of adjacent atoms being generated based on a topological connection of the atoms.

18. The atomic sequence rearrangement method according to claim 5, wherein the judgment of equivalent atoms step comprises: judging topologically equivalent atoms through a list of adjacent atoms, and the list of adjacent atoms being generated based on a topological connection of the atoms.

19. The atomic sequence rearrangement method according to claim 3, wherein the second rearrangement step comprises: performing rearrangement of the original structure with atomicity information and the reference structure for a second time, obtaining a structure consistent with the atomic sequence of the reference structure.

20. The atomic sequence rearrangement method according to claim 2, wherein the second rearrangement step comprises: performing rearrangement of the original structure with atomicity information and the reference structure for a second time, obtaining a structure consistent with the atomic sequence of the reference structure.
